(19) United States
(12) Patent Application Publication
(10) Pub. No.: US 2014/0316333 A1
(43) Pub. Date: Oct. 23, 2014

(54) DISSOLVING SOLID SOLUTION PERFORATOR PATCH FOR MIGRAINE TREATMENT

(71) Applicant: Sung-Yun KWON, Fremont, CA (US)

(72) Inventor: Sung-Yun Kwon, Fremont, CA (US)

(21) Appl. No.: 14/354,090

(22) PCT Filed: Oct. 29, 2012
(86) PCT No.: PCT/US12/62499

§ 371 Date: Apr. 24, 2014

Related U.S. Application Data
(60) Provisional application No. 61/552,792, filed on Oct. 28, 2011.

(51) Int. Cl. A61M 37/00; A61K 31/4468; A61K 31/137; A61K 31/485; A61K 38/26; A61K 31/4985; A61K 31/4535

(52) U.S. Cl. CPC A61M 37/0015; A61K 31/4985; A61K 31/4468; A61K 31/4535; A61K 31/485; A61K 38/26; A61K 31/137; A61M 2037/0023; A61M 2037/0046
USPC 604/46; 514/250; 514/331; 514/326; 514/282; 514/11.7; 514/653; 424/443

(57) ABSTRACT

A dissolving solid solution perforator (SSP) patch for oral cavity administration may include at least one perforator. The at least one perforator may contain a first drug and be configured to pierce an outside layer of an oral cavity for promptly delivering the first drug. The at least one perforator may penetrate an epithelium layer of the oral cavity and to deliver the antimigraine drug into blood vessels in a submucosa layer.

### CROSS REFERENCE TO PRIOR APPLICATIONS

The present application claims priority under 35 U.S.C. §119(e) to U.S. Provisional Application No. 61/552,792 (filed on Oct. 28, 2011), which is hereby incorporated by reference in its entirety.

The subject matter of this application is related to U.S. patent application Ser. No. 11/991,682, filed Apr. 28, 2008, as Attorney Docket No.: (840.0001), U.S. patent application Ser. No. 13/364,438, filed Feb. 2, 2012, as Attorney Docket No.: (840.0002), U.S. patent application Ser. No. 11/285,669, filed Nov. 21, 2005, as Attorney Docket No.: (840.0004), U.S. patent application Ser. No. 10/767,359, filed Jan. 28, 2004, as Attorney Docket No.: (840.0006), and U.S. patent application Ser. No. 10/179,749, filed Jun. 25, 2002, as Attorney Docket No.: (840.0003), the teachings of which are incorporated herein their entirety by reference.

### TECHNICAL FIELD

The present invention relates to a dissolving solid solution perforator (SSP) patch, and more particularly, to a dissolving SSP patch for treating migraine headache.

### BACKGROUND ART

Migraine is the underlying disorder of a brain function, usually manifesting as sensory hyper reactivity, which leads to periodic headache or other neurological disturbances. In the U.S., about 21 million women have problems with migraine headache and about 10% of the population has migraine headaches at least occasionally. Migraine is more prevalent than diabetes, epilepsy, asthma, and combined. The migraine headache normally lasts for about 4 to about 72 hours. Migraine sufferers are usually symptom free between headaches but may concern the fear of the next attack.

Symptoms of migraine headache may include prodrome, aura, and/or headache. The prodrome phase of migraine can occur several hours or days before a major headache. During the prodrome phase, a patient may experience the following symptoms: depression, hyperactivity, euphoria, irritability, sensitivity to light or sound, diarrhea or constipation, and drowsiness. The aura phase is not experienced by all migraineurs. The aura phase typically precedes the headache by an hour or less. Most aura symptoms last between 15 and 60 minutes. The aura phase may be characterized by visual, sensory, or motor symptoms and may also involve disturbances in language. The most common aura symptoms are visual. For example, the common aura symptoms include the appearance of flashes, specks, zigzag lines, stars, or shimmery areas. Furthermore, blind spots or tunnel vision may also occur. Less common aura symptoms involve speech disturbances, confusion, tingling or numbness, weakness of the limbs, and confusion. The headache phase may last for several hours or for three days in adults. The headache phase may be characterized by throbbing pain on one side of head or the whole head. Most migraineurs experience nausea, with or without vomiting. Most migraineurs are extremely sensitive to light and noise. A person suffering from a migraine may be pale and sweaty and has cold hands and feet. In addition, such person, a migraineurs, has symptoms of visual disturbances, faintness, diarrhea, a stiff or tender neck, and aversion to food.

The detailed mechanism of migraine headache is still a subject for research and study. It is, however, generally believed that migraines are caused by a rapid widening and narrowing of blood vessel walls in the brain and head. Certain factors have been identified, which trigger migraine headache in susceptible people. The certain factors may be stress, the relief of stress, lack of food or infrequent meals, foods containing monosodium glutamate (MSG), tyramine (certain specific foods like chocolate) citrus fruits or cheese, alcohol (especially red wine), overtiredness (physical or mental), changes in sleep patterns (e.g., late nights or a weekend lie-in), or hormonal factors (e.g., monthly periods, the contraceptive pill or hormonal changes in males and females as they age), etc.

Since migraine is a complex symptom, a successful treatment for one patient may have no effect on the other. Ongoing research centers on new methods of treatment and new administration method that show therapeutic potential in mitigating migraines.

Management of migraine is complicated because of the lack of a single, effective therapy in all patients. For treating the same migraine type, we need to select either an abortive or prophylactic method of treatment for these migraines. Furthermore, complications involve the current use of drugs that cause dependence with extended use. Another important consideration is that the more effective antimigraine agents in current use produce severe use-limiting side effects with long term usage. Thus, there is a need for a general and effective administration for the treatment and related disorders which can be used either prophylactically or to alleviate an established migraine.

A typical method of oral administering dihydroergotamine (DHE) to migraine sufferers has major efficacy limitations. Oral dosage of DHE is generally in large doses of about 20-30 mg due to degradation in the gastrointestinal tract and low adsorption of the drug. These high doses lead to nausea, vomiting and undesired adverse side effects. It is estimated that about 2-10% of the active unchanged drug actually reaches the blood stream. Also oral dosage takes 2-3 hours to have efficacy for reducing migraine headache. To have fast efficacy, nasal spray dosage is available but has significant limitations too. A 2 mg intranasal dose of a pharmaceutical salt of DHE must be administered as 4 intranasal sprays with subsequent reduced systemic absorption by the unintended oral ingestion of DHE intranasal solution. Injectable forms of DHE are also available for the treatment of migraines. Although parenteral administration of DHE into the blood stream allows for a lower dose as compared to other non-injectable methods of administration, the inconvenience of an office visit for an injection and the potential problems with the self-administration of injectable are self-evident. Basically, similar limitations apply in the use of triptan.

### DISCLOSURE

### Technical Problem

Embodiments of the present invention overcome the above disadvantages and other disadvantages not described above. Also, the present invention is not required to overcome the disadvantages described above, and an embodiment of the present invention may not overcome any of the problems described above.

In accordance with an aspect of the present invention, a dissolving solid solution perforator (SSP) patch containing antimigraine agents may be provided for effectively and quickly treating migraine headache.

In accordance with another aspect of the present invention, a dissolving SSP patch may allow oral cavity administration for effective and rapid treatment of migraine headaches. Such administration method may enable the dissolving SSP patch to treat migraine with comparatively less doses of therapeutic compound, thereby minimizing side effects thereof.

In accordance with still another aspect of the present invention, a dissolving SSP patch may contain active ingredients such as triptans or dihydroergotamine (DHE) for treating headaches in fast onset, higher efficacy, and fewer side effects.

In accordance with yet another aspect of the present invention, a dissolving SSP patch may have bio-adhesive properties that enable the dissolving SSP patch to adhere and bond with musical tissue when the dissolving SSP patch is applied on a buccal mucosa.

In accordance with yet another aspect of the present invention, a dissolving SSP patch may be formed of fast-dissolving and/or swelling material.

In accordance with yet another aspect of the present invention, a dissolving SSP patch may include a reservoir layer containing a supplementary drug. The reservoir layer may be included in a basal layer and the supplementary drug might be the same or different from a primary drug contained in at least one of perforators.

In accordance with yet another aspect of the present invention, a dissolving SSP patch may be provided for prophylactic and acute treatment of migraine.

### Technical Solution

In accordance with an embodiment of the present invention, a dissolving solid solution perforator (SSP) patch for oral cavity administration may include at least one perforator. The at least one perforator may be configured to contain a first drug and to pierce an outside layer of an oral cavity for promptly delivering the first drug. The at least one perforator may be configured to penetrate an epithelium layer of the oral cavity and to deliver the first drug into a submucosa layer.

The at least one perforator may be made of a dissolvable biocompatible material strong enough to pierce the epithelium layer and provides bio-adhesion to the oral cavity. For example, the at least one perforator may be fabricated with a sodium carboxy methyl cellulose (Na-CMC). The dissolving SSP patch may include about 20 to 100 perforators in an area of about 1 cm2.

The at least one perforator may be configured to have an elongated structure sufficiently long to penetrate the outside layer of the oral cavity, to deliver the first drug to blood vessels in a submucosa layer, and to prevent damaging the blood vessels in the submucosa layer. A length of the at least one perforator may be in a range of about 700 to 1800 μm. A length of the at least one perforator may be shorter than 2000 μm. The at least one perforator may be configured to adhere to the oral cavity until the at least one perforator is completely dissolved and the first drug is completely delivered.

The first drug may include antimigraine drug. For example, the first drug may include at least one of triptans and dihydroergotamine (DHE). Furthermore, the first drug may include at least one of analgesics and anesthetics. For example, the first drug includes non-steroidal anti-inflammatory drugs (NSAID). In addition, the first drug may include at least one of fentanyl, sufentanil, oxycodone, hydromorphone, morphine, glucagon, and epinephrine.

In accordance with another embodiment of the present invention, the dissolving SSP patch may further include a basal layer. The basal layer may be formed on one end of the at least one perforator and configured to provide instant mucosal adhesion to the oral cavity. The basal layer may include a reservoir containing a second drug. The second drug may be the same as the first drug or different from the first drug. The second drug may be at least one of antivirus drug and anti-bacterial protection drug for suppressing infection.

In accordance with still another embodiment of the present invention, the dissolving SSP patch may further include a backing layer. The backing layer may be formed on the basal layer to cover one side of the basal layer and configured to protect the at least perforator from local saliva and tongue movement. The baking layer may contain flavor and color components to mask medicine taste. The backing layer may be fabricated with a dissolvable and edible material. The backing layer may be fabricated with a material different from materials of the at least one perforator and the basal layer. The backing layer may dissolve slowly than the at least one perforator and the basal layer.

### Advantageous Effects

A dissolving solid solution perforator (SSP) patch may effectively treat migraine headache with comparatively small doses by delivering a migraine drug through buccal administration. Accordingly, the dissolving SSP patch may maximize a degree of absorbing migraine drug in a target brain area within therapeutic level and minimize side effects.

### DESCRIPTION OF DRAWINGS

The above and other aspects, features and other advantages of the present invention will be more clearly understood from the following detailed description taken in conjunction with the accompanying drawings, in which:

FIG. 1 is a cross-sectional view of a buccal mucosa;

FIG. 2A shows a dissolving SSP patch in accordance with at least one embodiment of the present invention;

FIG. 2B shows a magnified view of a part of a dissolving SSP patch of FIG. 2A;

FIG. 3 is a scanning electric microscopic (SEM) image of a dissolving SSP patch in accordance with at least one embodiment of the present invention;

FIG. 4 shows a dissolving SSP patch applied on oral mucosa in accordance with at least one embodiment of the present invention;

FIG. 5 is a graph showing a result of delivering an antimigraine drug via a buccal membrane using a dissolving SSP patch in accordance with an embodiment of the present invention;

FIG. 6 shows a fabrication method for a dissolving SSP patch in accordance with at least one embodiment of the present invention;

FIG. 7 shows a mold for a dissolving SSP patch in accordance with at least one embodiment of the present invention.

### BEST MODE

Reference will now be made in detail to embodiments of the present invention, examples of which are illustrated in the accompanying drawings, wherein like reference numerals refer to like elements throughout. The embodiments are described below, in order to explain the present invention by referring to the figures.

The practice of the present invention will employ, unless otherwise indicated, conventional methods of engineering, chemistry, biochemistry, pharmacology, and drug delivery, within the skill of the art. Such techniques are explained fully in the literature. All publications, patents and patent applications cited herein, whether supra or infra, are hereby incorporated by reference in their entirety.

In accordance with at least one embodiment of the present invention, a dissolving solid solution perforator (SSP) patch may be provided for effectively treating migraine with comparatively less required doses. The dissolving SSP patch may contain antimigraine agent such as DHE, triptan and/or pharmaceutical salt with a migraine effective dose amount. Such a dissolving SSP patch may be configured to allow buccal administration, oral cavity administration, and/or trans-oral administration of the antimigraine agents. Accordingly, the dissolving SSP patch may effectively inject active ingredients of antimigraine drugs into a blood stream, thereby maximizing a degree of absorbing DHE or triptan in a target brain area within therapeutic level and minimizing side effects. That is, the dissolving SSP patch may allow injection of DHE and/or triptan through an oral mucosa by buccal administration and oral cavity administration in accordance with at least one embodiment of the present invention. Since DHE and/or triptan are absorbed through oral mucosa, migraine headache may be effectively and rapidly treated with comparatively low doses, as compared to doses used for typical administration. Such low dose treatment of migraine headache may reduce adverse effects. Furthermore, the dissolving SSP patch may allow injection of antimigraine agent through a trans-buccal route and a trans-oral route in accordance with embodiments of the present invention. Accordingly, the dissolving SSP patch may allow the ease of administration and inject the antimigraine agent in an effective route, which is close to a blood vessel to brain.

The dissolving SSP patch may be referred to as a microneedle, a microneedle array, a microneedle system, and/or a microneedle SSP system. Although the dissolving SSP patch is described throughout the specification as having a configuration for buccal administration and trans-oral administration, the present invention is not limited thereto.

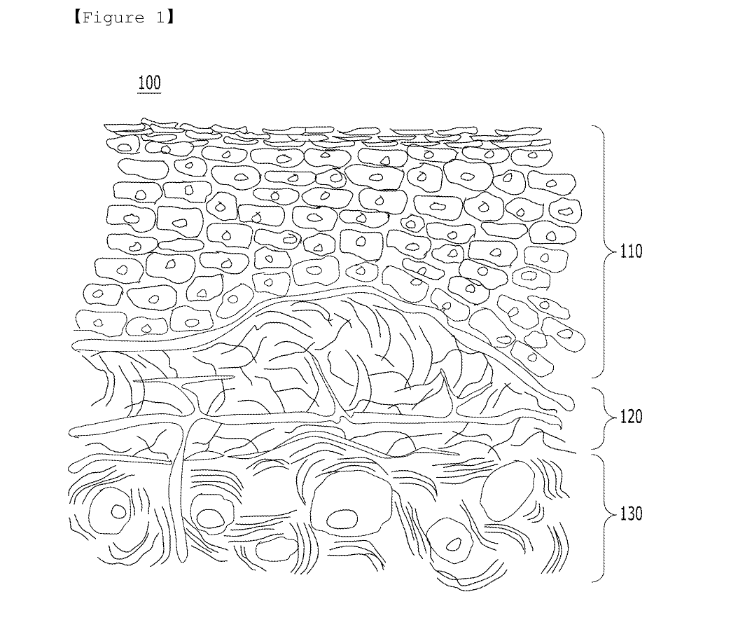

Hereinafter, a dissolving SSP patch and a method of administering antimigraine agent using the radio dissolving SSP patch in accordance with embodiments of the present invention will be described with reference to FIG. 1 to FIG. 4. As described, the dissolving SSP patch in accordance with at least one embodiment of the present invention allows a buccal administration and/or an oral cavity administration of an antimigraine drug. That is, the dissolving SSP patch may be applied on oral mucosa to administer the antimigraine agent. The buccal administration and/or the oral cavity administration of the dissolving SSP patch will be described with reference to FIG. 1.

FIG. 1 is a cross-sectional view of a buccal mucosa.

The buccal mucosa is mucous membranes lining an inside of a mouth. The buccal mucosa may be referred to as an oral mucosa. As shown in FIG. 1, buccal mucosa 100 may include epithelium 110, lamina propria 120, and submucosa 130. Buccal mucosa 100 may further include a basement membrane between epithelium 110 and lamina propria 120.

Epithelium 110 may be a stratified squamous layer. Epithelium 110 may include a permeability barrier at the outermost portion thereof. A thickness of such a permeability barrier is about 200 μm. The permeability barrier may be a result of intercellular material derived from the so-called ‘membrane coating granules’ (MCG).

Aside from the permeability barrier such as the MCGs, the basement membrane between epithelium 110 and lamina propria 120 may be an additional permeability barrier that acts as resistance to permeation as well. However, the outer epithelium is still considered to be the rate limiting step to mucosal penetration. The structure of the basement membrane is typically not dense enough to exclude even relatively large molecules and is more tolerable to microneedle insertion than skin. At least one perforator of the dissolving SSP patch in accordance with at least one embodiment of the present invention may penetrate the permeability barriers of buccal mucosa 100 and form a channel to deliver the antimigraine agents contained therein to blood vessels under the epithelium 110 and lamina propria 120.

As descried, epithelium 110 and lamina propria 120, which are an epidermis or epidermal layer, generally contain no blood vessels. Such epidermis freely exchanges metabolites by diffusion to and from submucosa 130. Submucosa 130 may be located immediately below the epidermis. Below the epidermis, submucosa 130 is present, which may be referred to as a dermis or a dermal layer. The thickness of the dermis is about 1 to 3 mm. The dermis contains blood vessels, lymphatics, and nerves. Once the dissolving SSP patch delivers the antimigraine agents to the dermal layer, the antimigraine agents perfuse through system circulation.

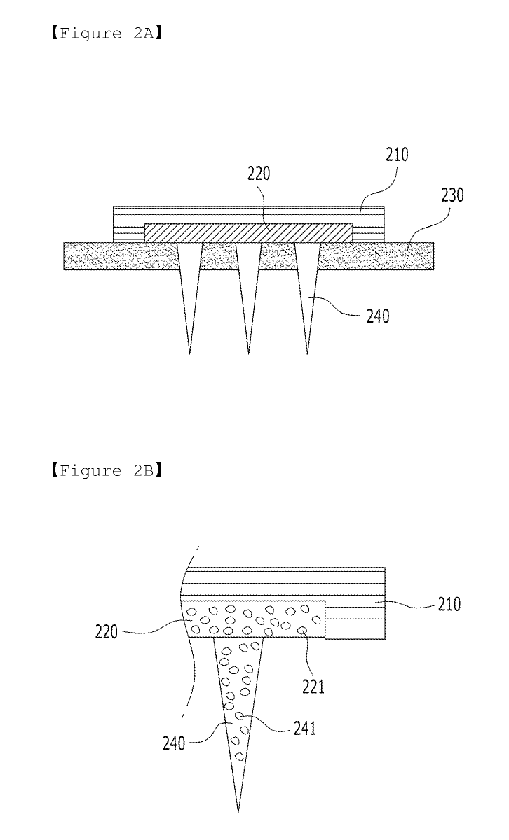

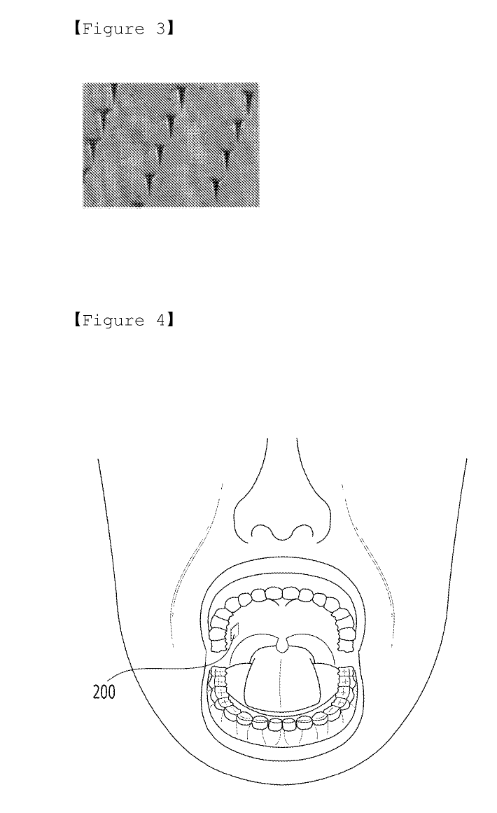

As described, the dissolving SSP patch in accordance with at least one embodiment of the present invention is applied on a predetermined area of buccal mucosa 100 for treating migraine headache. The dissolving SSP patch may penetrate the epidermis of buccal mucosa 100, form a channel to the dermis, and deliver the migraine agents to blood vessels. Since the dissolving SSP patch is applied on the less dense oral cavity, such a comparatively large molecular weight of the migraine agent is quickly delivered to the blood vessel to a predetermined area of brain without excessive pain and bleeding. Hereinafter, the dissolving SSP patch in accordance with at least one embodiment of the present invention will be described with reference to FIG. 2.

FIG. 2A shows a dissolving SSP patch in accordance with at least one embodiment of the present invention. FIG. 2B shows a magnified view of a part of dissolving SSP patch of FIG. 2A. FIG. 3 is a scanning electron microscopic (SEM) image of a dissolving SSP patch in accordance with at least one embodiment of the present invention.

Referring to FIG. 2A, dissolving SSP patch may include backing layer 210, basal layer 220, and a plurality of perforators 240 in accordance with at least one embodiment of the present invention. As shown, dissolving SSP patch may include at least one perforator 240, which may be referred to as a microneedle. For example, dissolving SSP patch may include at least 20 to 500 perforators 240 in an area of about 1 cm² in accordance with an embodiment of the present invention. Preferably, dissolving SSP patch may include 20 to 100 perforators in an area of about 1 cm2.

Perforator 240 may contain antimigraine drug in accordance with at least one embodiment of the present invention. For example, active ingredients (solid form of DHE or triptan) are contained in perforator 240. The primary functions of perforator 240 may be to pierce the outside of layered epithelium 110, to provide prompt initiation drug delivery, and to adhere to the oral cavity tissue until perforator 240 and/or dissolving SSP patch is completely dissolved and all drug in perforator 240 and/or dissolving SSP patch is delivered. Perforator 240 may help keep a channel open for subsequent drug delivery until a micro channel is closed and a portal channel likely will contract or expand depending on material properties of perforator 240 after perforator 240 and/or dissolving SSP patch dissolves or swells.

Perforator 240 may be formed as a solid matrix. Perforator 240 may be strong and intact enough to pierce an outside squamous stratified epithelium layer. Perforator 240 may be made of a dissolving matrix providing instant bio-adhesion to the oral cavity tissue. Accordingly, perforator 240 and/or dissolving SSP patch 200 may start to dissolve when perforator 240 including the antimigraine drug has penetrated into the target tissue, such as the oral cavity tissue. Perforator 240 and/or dissolving SSP patch 200 may be dissolved and swallowed in a comparatively short time intervals. For example, perforator 240 may be dissolved and swallowed in between a few tens of seconds and several hours. As long as perforator 240 and/or SSP patch 200 dissolves reasonably quickly and is strong enough to pierce the epithelium, fundamentally any biocompatible material may serve as a material for perforator 240 and/or SSP patch 200. For example, a sodium carboxy methyl cellulose (Na-CMC) may be used as the material for forming perforator 240 and/or SSP patch 200 because Na-CMC is inert and suitable for microneedle and fabrication.

Perforator 240 may have a sharpened end for perforating on an oral cavity tissue, for example buccal mucosa 100. Perforator 240 may have at least one of shapes of a straight shaft, a tapered shaft, a pyramid, a wedge, a needle, and a blade. The present invention, however, is not limited thereto. Perforator 240 may have any other shapes that penetrate the outside surface of an oral cavity tissue.

Perforator 240 may have an elongated structure that is sufficiently long to penetrate through the outmost barrier. The length of perforator 240 may be in a range of about 1 to 2000 μm. Particularly, the length of each microneedle 240 may be in a range of about 700 to 1800 μm. A buccal tissue is not a smooth and rugged surface and has different depths microscopically. In addition, the thickness of epithelium 110 and elasticity of the oral cavity tissue varies. Accordingly, the length of perforator 240 may have an enough length to penetrate an oral cavity tissue and deliver the migraine agents to blood vessels under epithelium 110 and lamina propria 120. A desirable penetration depth has a range, rather than a single value, for effective drug delivery and for painless and bloodless penetration. The penetration depth of penetrator 240 may affect pain as well as delivery efficiency. In accordance with at least one embodiment of the present invention, the penetration depth of perforator 240 may be less than 2000 μm so that perforator 240, inserted into the buccal tissue through the most outside layer of epithelium 110, does not penetrate past submucosa 130. Accordingly, perforator 240 may not contact nerves and blood vessels.

As shown in FIG. 2A, dissolving SSP patch 200 may further include basal layer 220 and backing layer 210 in accordance with at least one embodiment of the present invention.

Basal layer 220 may provide instant mucosal adhesion to the oral cavity tissue. Furthermore, basal layer 220 may provide a reservoir of drugs like DHE or triptan in accordance with at least one embodiment of the present invention. The function of basal layer 220 is adhesion to the oral cavity tissue and provide extra drug for sustained delivery. A thickness of basal layer 220 may vary. Where additional and sustained drug release is required, basal layer 220 may be constructed to contain more of a drug. For example, basal layer 220 may contain second drug 221 such as antivirus and/or anti-bacterial protection drug to suppress infection, as shown in FIG. 2B. As described, such second drug 221 may be different from first drug 241 contained in perforator 240, but the present invention is not limited thereto. In some embodiment, second drug 221 may be the same drug as first drug 241 contained in perforator 240. Although first and second drugs 221 and 241 are illustrated as drug particles, the present invention is not limited thereto. For example, first and second drugs 221 and 241 may be a solid form and a semisolid form.

Backing layer 210 may be formed on basal layer 220 in accordance with at least one embodiment of the present invention. For example, backing layer 210 may cover at least one outer side of basal layer 220. Backing layer 210 may provide protection of perforator 240 from local saliva and tongue movement. Backing layer 210 may contain flavor and color components to mask medicine taste. Backing layer 210 may be made of material that is a dissolvable and edible matrix, but the present invention is not limited thereto. In some embodiment, backing layer 210 may be formed of un-dissolvable matrix depending on application.

Backing layer 210 may be formed of material different from that of perforator 240 and/or basal layer 220 in order to help perforator 240 and/or basal layer 220 to fully dissolve. For example, backing layer 210 may be formed of material dissolving slowly than perforator 240 and basal layer 220. Backing layer 210 may be prepared by at least one of direct compression, dry granulation, and wet granulation. After forming backing layer 210, backing layer 210 may be combined with basal layer 220 and perforator 240. For example, backing layer 210 may be bonded on a base layer of perforator 240.

Dissolving SSP patch 200 may further includes a reservoir that contains solid or semi-solid form of a supplementary drug. Such reservoir may be intended to provide sustained and controllable delivery of the antimigraine drug such as DHE and triptan into or across a biological barrier so that diffusion channels are created and remain open after insertion and dissolution of perforator 240 including the primary drug. For example, a reservoir may be included in at least one of basal layer 220 and perforator 240, but the present invention is not limited thereto. Such a reservoir may be formed in a shape of a layer and included between perforator 240 and basal layer 220 and/or between basal layer 220 and baking layer 240. Furthermore, the reservoir may be formed in material different from that of at least one of perforator 240, basal layer 220, and backing layer 210 in order to control the delivery of the primary drug and the supplementary drug. Alternatively, the reservoir may be formed in the same material of at least one of perforator 240, basal layer 220, and backing layer 210. In addition, the supplementary drug may be approximately or substantially identical to the primary drug contained in perforator 240. For example, the supplementary drug may be antimigraine drug such as DHE and triptan, but the present invention is not limited thereto. In some embodiment, the supplementary drug may be different from the primary drug. For example, the supplementary drug may be antivirus and/or anti-bacterial protection drug.

As shown in FIG. 2A and FIG. 2B and described, dissolving SSP patch 200 may a rectangular shape, a certain size, and compositions, but the present invention is not limited thereto. Such shape, a size, a composition, and an areal density of dissolving SSP patch 200 may affect a drug release rate. Accordingly, the shape, the size, the composition, and the areal density of dissolving SSP patch 200 may vary according to a desired drug release rate.

Dissolving SSP patch 200 was described as containing at least one of DHE and triptan. The present invention, however, is not limited thereto. In some embodiments, dissolving SSP patch 200 may include at least one of: beta-blocking agents, calcium channel blocking agents, antidepressants, selective 5-HT 1 agonists (triptan), sedatives, local anesthetics, adrenergic blocking agents and mixtures of these. Furthermore, dissolving SSP patch 200 may also include at least one of methysergide maleate, propranolol hydrochloride, ergotamine tartrate, a vasoconstrictor, amitriptyline, an antidepressant (valproic acid), an anticonvulsant, verapamil, and a calcium channel blocker.

FIG. 4 shows a dissolving SSP patch applied on oral mucosa in accordance with at least one embodiment of the present invention.

As shown in FIG. 4, dissolving SSP patch 200 in accordance with at least one embodiment of the present invention may be placed on a patient's mouth and holding it in the mouth, either adjacent a cheek and/or between the upper lip and gum. That is, the dissolving SSP patch may deliver the antimigraine drug through buccal administration. The dissolving SSP patch begins to dissolve or disintegrate due to the moisture in the mouth and saliva. Such a delivery of antimigraine drugs via oral cavity such as buccal and sublingual is a very effective for achieving systemic or vaccination effects.

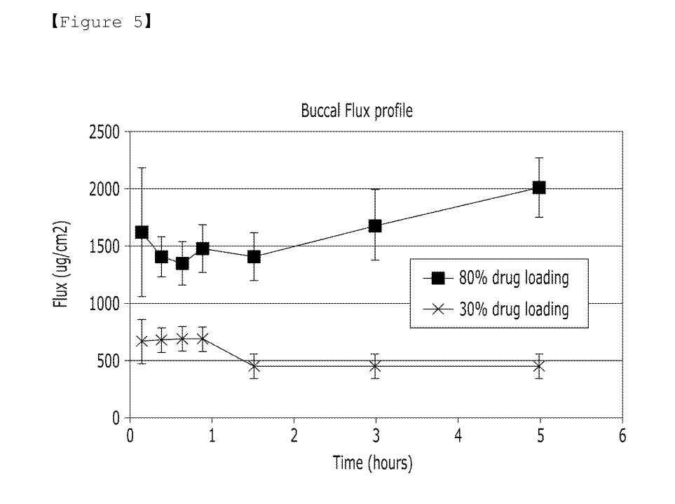

FIG. 5 is a graph showing a result of delivering an antimigraine drug via a buccal membrane using a dissolving SSP patch in accordance with an embodiment of the present invention.

Referring to FIG. 5, the graph shows buccal flux profile obtained through vitro flux experiments. In the vitro flux experiments, sumatriptan may be used as an antimigraine drug. The sumatriptan was injected using dissolving SSP patch 200 and buccal flux profile of the sumatriptan was measured. The sumatriptan was prepared as follows: i) trehalose and CMC were dissolved in D.L water with a predetermined ratio to form gel; ii) the gel was cast into a mold by centrifugation for 5 minutes and dried under ambient conditions; and iii) perforator matrix was separated from the mold and cut into 1 cm² discs each having 45 perforators when the gel is fully dried.

For the vitro flux experiments, a modified Franz cell was used with pig buccal membrane and D. I. water (deionized) as receiver medium. At predetermined intervals such as 15, 30, 45, 60 minutes and 2, 4 and 6 hours, entire receiver volumes were collected from the receiver of the diffusion cells and fresh receiver media were refilled. The samples were assayed for sumatriptan content by high-performance liquid chromatography (HPLC).

In the vitro flux experiments, perforators of a SSP patch may be composed of 24% sumatriptan, 6% trehalose and 70% Na-CMC displayed initial flux of 700 ug/cm2/h. With the SSP patch of 80% drug loading formulation, 1700 ug/cm2/h was delivered as shown in the graph of FIG. 5. The buccal flux profile of FIG. 5 shows that the SSP patch in accordance with at least one embodiment of the present invention delivers high flux. For example, the SSP patch may deliver up to total 6 mg of sumatriptan in 4 hours, which is quite promising as a delivery amount for more potent drugs.

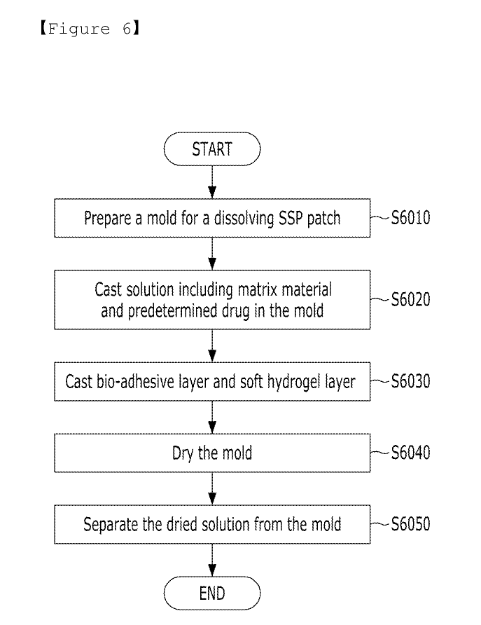

FIG. 6 shows a fabrication method for a dissolving SSP patch in accordance with at least one embodiment of the present invention.

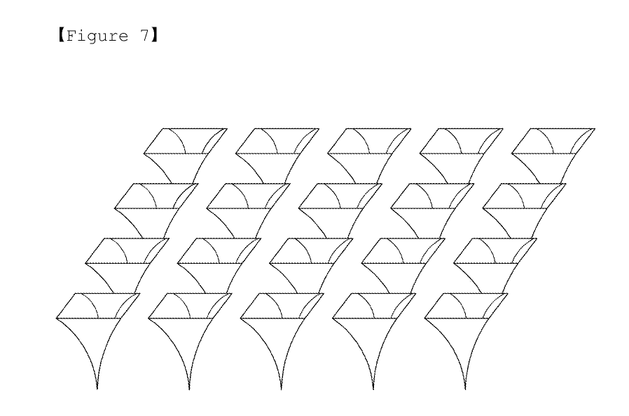

Referring to FIG. 6, a mold for a dissolving SSP patch may be prepared at step S6010. For example, the mold of FIG. 7 may be prepared through precision machining such as milling, micro-machining (such as MEMS), laser-based machining, and electro-discharge machining. For a description of representative mold, see e.g., U.S. patent application Ser. No. 13/364,438, filed Feb. 2, 2012, as Attorney Docket No.: (840.0002), incorporated herein by reference in its entirety.

At step S6020, solution including a matrix material and a predetermined drug may be cast in the mold and dried. The solution may be at least one of liquid, gel solution, and melted sugar. The predetermined drug may be an antimigraine drug such DHE or triptan. The present invention, however, is not limited thereto. Instead of casting such active ingredient solution on the mold, an accurate dispenser such as an inject style dispenser may be used. In this case, the active ingredient solution may be dispensed on each perforator mold. In accordance with at least one embodiment of the present invention, Na-CMC fills the mold with the accurate dispenser. For a description of preparing and casting the solution including the predetermined drug, see, e.g., U.S. patent application Ser. No. 11/991,682, filed Apr. 28, 2008, as Attorney Docket No.: (840.0001), incorporated herein by reference in its entirety.

At step S6030, a bio-adhesive layer and a soft hydrogel layer may be sequentially cast after the filling. For example, the bio-adhesive layer may be a basal layer and the soft hydrogel may be a backing layer. Depending on the viscosity and other physical and chemical properties of the liquid solution, additional force such as centrifuge force or compression force may be applied.

At step S6040, the mold may be dried to form a solid solution. For example, in order to dry, at least one of air dry, vacuum dry, and freeze dry may be applied.

At step S6050, once the solution is completely dried, the dried solution may be separated from the mold and cut to an appropriate shape and size for oral cavity administration. The shape and size may vary according to a desired drug release rate. In accordance with at least one embodiment of the present invention, a size of the dissolving SSP patch may be about 1 to 2 cm2.

As described, melted sugar may be used as a material for the SSP patch. In this case, sugar including the predetermined drug may be melted at a temperature lower than a decomposed temperature. For example, sucrose may be melted at about 160° C. Such melted sucrose including the predetermined drug may be cast in the mold and cooled. In case of using an accurate dispenser such as an inkjet style dispenser, the solution is accurately dispensed on each perforator mold. The solution in the mold may be cooled in ambient condition. It may be sufficient to rapidly form a perforator.

Furthermore, a powder form may be used for the material for the SSP patch. In this case, mixed powder may be spread over the mold. For example, the mixed power may include a predetermined drug particle. Depending upon chemical and physical properties of the mixed powder, a direct compression process, a wet granulation process, and a heating process may be applied to melt the mixed power and to insert viscous material into the mold. Alternatively, the mixed powder may be inserted into the mold by pressure and/or application of heating with use of binding agents.

In at least one embodiment of the present invention, analgesics or anesthetics may be added to pharmaceutical preparation to alleviate any side effects of the medication or of the migraine. Furthermore, in some embodiment, non-steroidal anti-inflammatory drugs (NSAID) may be added as additional active ingredients. The present invention, however, is not limited to any particular type of NSAID.

Since the SSP patch in accordance with at least one embodiment of the present invention is configured for the buccal administration, the SSP patch may be used for breaking through pain management and emergency application. For breakthrough pain management, fentanyl, sufentanil, oxycodone, hydromorphone, morphine may be added into the SSP patch. For emergency application, glucagon or epinephrine may be added into the SSP patch.

As described, the dissolving SSP patch may be fabricated using polymer matrix materials. For example, such polymer matrix materials may include polyvinylpyrolidone (PVP), polyethylene glycol (PEG), polyethylene oxide (PEG), polyvinyl alcohol (PVA), cellulose, hydroxypropyl cellulose (HPC), hydroxyethyl cellulose (HEC), hydroxypropyl methylcellulose (HPMC), pectin, dextrin, mono-and polysaccharide, sodium carboxymethyl cellulose (Na-CMC), polyalcohol, gelatin, gum arabic, cellulose gum, alginate, chitosan cylcodextrin, Cabopol and other biopolymers. The present invention, however, is not limited thereto. Particularly, dissolving SSP patch 200 may be fabricated using Na-CMC, where molecular weight is ranged from 20,000 to 250,000.

Furthermore, such matrix materials may include carbohydrate derivatives, such as sugar derivatives. For example, the sugar derivatives may include trehalose, glucose, maltose, lactose, maltulose, iso-maltulose, lactulose, raffinose, mannose, fluctose, turanose, melitose, melezitose, dextran, maltotol, sorbitol, inositol, xylitol, palatinit and mannitol. For forming the matrix materials, a melt-and-cooling method may be suitable. Also these sugars can be mixed with the matrix polymer.

Water-soluble ingredients, such as phosphate, nitrate and carboxylate glasses, magnesium chloride, potassium chloride and calcium chloride may be also used for a matrix material, alone or mixed with a matrix polymer. Adding sugar derivatives can prompt dissolution, reduce dissolution time and modify the hardness of SSP. In order to enhance permeation, tissue reaction or fabrication, some surfactant can be beneficial.

In addition, suitable matrix materials may further include at least one of non-ionic hydrophilic, ionic surfactants, lipophilic additives. The suitable matrix materials may include alkylglucosides, alkylmaltosides, alkylthioglucosides, lauryl macrogolglycerides, polyoxyethylene alkyl ethers, polyoxyethylene alkylphenols, polyethylene glycol fatty acids esters, polyethylene glycol glycerol fatty acid esters, polyoxyethylene sorbitan fatty acid esters, polyoxyethylenepolyoxypropylene block copolymers, polyglycerol fatty acid esters, polyoxyethylene glycerides, polyoxyethylene sterols, derivatives, and analogues thereof, polyoxyethylene vegetable oils, polyoxyethylene hydrogenated vegetable oils, reaction mixtures of polyols and at least one member of the group consisting of fatty acids, glycerides, vegetable oils, hydrogenated vegetable oils, and sterols, tocopherol polyethylene glycol succinates, sugar esters, sugar ethers; sucroglycerides, and mixtures thereof.

The ionic surfactant may include: alkyl ammonium salts; bile acids and salts, analogues, and derivatives thereof; fatty acid derivatives of amino acids, carnitines, oligopeptides, and polypeptides; glyceride derivatives of amino acids, oligopeptides, and polypeptides; acyllactylates; mono-diacetylated tartaric acid esters ofmono-diglycerides; glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The lipophilic additive may include alcohols, polyoxyethylene alkylethers, fatty acids, bile acids, glycerol fatty acid esters, acetylated glycerol fatty acid esters, lower alcohol fatty acids esters, polyethylene glycol fatty acids esters; polyethylene glycol glycerol fatty acid esters, polypropylene glycol fatty acid esters, polyoxyethylene glycerides, lactic acid derivatives of mono/diglycerides, propylene glycol diglycerides, sorbitan fatty acid esters, polyoxyethylene sorbitan fatty acid esters, polyoxyethylene-polyoxypropylene block copolymers, transesterified vegetable oils, sterols, sterol derivatives, sugar esters, sugar ethers, sucroglycerides, polyoxyethylene vegetable oils, polyoxyethylene hydrogenated vegetable oils, reaction mixtures of polyols and at least one member of fatty acids, glycerides, vegetable oils, hydrogentated vegetable oils, and sterols, and mixtures thereof.

The addition of surfactants may be used in the manufacturing process for easy casting and filling the mold depending on the mold materials by adjusting surface tension of the mold surface.

The chemical enhancer may be a fatty alcohol, an acid, an ester, a surfactant, a macrocyclis, a terpene, a phospholipid, a pyrrolidone, an amide or an amino acid. More particularly, a chemical enhancer may be drawn from among alkyl alcohol, alpha bisabodol, decyl alcohol, dexpanthenol, dodecanol, etylene glycol, fatty alcohols, glycerol, hexadecanol, isopropanol, octadecanol, tetrahydrofurfuryl alcohol, trichloroethanol, trifluoroethanol, alkyl acetamide, crotamiton, lauryl diethanolamide, toluamide, dimethyl acetamide, dimethyl formide, formamide, nicotainamide, acyl-amino-acids, alanine, arginine, proline, serine, aspartic acid, cysteine, glutamic acid, glycine, valine, leucine, isoleucine, protein aprotinin, azone, essential oils, such as carvone, cineole, eucalyptol, eugenol, methol, methone, terpene fatty acids, such as carboxyl acid, capric acid, diisopropyladipate, isopropyl myristate (IPM), isostearic acid, glyceryl monolaurate (GML), glycerol monooleate (GMO), lactic acid, linoleic acid, lauric acid, methyllaurate, methyl myristate, oleic acid, polyethylene glycol monolaurate, sorbitan monooleate (SMO), sucrose cocoate, sucrose monoloaurate, sucrose monooleate, triglyceride, macrocyclic enhancers, such as cyclodextrin, cyclopentadecanone and cyclopentadecanolide, phospholipids, phospholipid/phosphate enhancers, such as dialkylphospahte, lecithin, dioxane, dioxolane, alkylsulfones, alkylsulfones, cetyl ether, cyclic dimethylsiloxane, decamethyltetrasiloxane, dialkyl sulfoxides, dimethylsulfoxide, decylmethylsulfoxide, hexamethyldisiloxane, methyl octylsulfoxide, alkyl ammonium bromide, benzyl nicotinate, butylazocyclopentane, capsaicin, calcium thioglycolate, cyclic amine, diethyl sebacate, dimethylamino acetate, ethylene glycol monoethyl ether, imidazole, methylorthoformate, oxazoline, proline, urea, urethane, macrocyclis, amines, alkyl pyrolidones, N-methylpyrrolidone, ethyl pyrrolidone, pyrrolidone, hydroxymethylpyrrolidone, hexyl pyrrolidone, lauryl pyrrolidone, pyrrolidone-carboxylic acid, lauryl pyrrolidone carboxylic acid, pyroglutamic acid, sodium dedecyl sulfate, sodium deoxycholate, sodium lauryl sulfate, sorbitan monopalmitate, sorbitan trioleate, soybean casein, terpenes, piperazine derivatives, sodium traurocholate, liposome, bisbolol, dithiothreitol and vitamin E (alpha.-tocopherol).

In some embodiment, an effervescent agent may be used. Furthermore, acid sources may be any sources that are safe for human consumption. Such acid source may include food acids, acid and hydrite antacids such as, for example: citric acid, tartaric, amalie, fumeric, adipic and succinics. Carbonate sources may include dry solid carbonate and bicarbonate salt such as sodium bicarbonate, sodium carbonate, potassium bicarbonate and potassium carbonate, magnesium carbonate and the like, but the present invention is not limited thereto. In addition, reactants that evolve oxygen or other gasses may be used. Any reactants safe for human consumption may be used.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles and “an” as used in this application and the appended claims should generally be construed to mean “one or more” unless specified otherwise or clear from context to be directed to a singular form.

Moreover, the terms “system,” “component,” “module,” “interface,”, “model” or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

As used herein in reference to an element and a standard, the term “compatible” means that the element communicates with other elements in a manner wholly or partially specified by the standard, and would be recognized by other elements as sufficiently capable of communicating with the other elements in the manner specified by the standard. The compatible element does not need to operate internally in a manner specified by the standard.

No claim element herein is to be construed under the provisions of 35 U.S.C. §112, sixth paragraph, unless the element is expressly recited using the phrase “means for” or “step for.”

Although embodiments of the present invention have been described herein, it should be understood that the foregoing embodiments and advantages are merely examples and are not to be construed as limiting the present invention or the scope of the claims. Numerous other modifications and embodiments can be devised by those skilled in the art that will fall within the spirit and scope of the principles of this disclosure, and the present teaching can also be readily applied to other types of apparatuses. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A dissolving solid solution perforator (SSP) patch for oral cavity administration, the dissolving SSP patch comprising:
at least one perforator configured to contain a first drug and to pierce an outside layer of an oral cavity for promptly delivering the first drug.

2. The dissolving SSP patch of claim 1, wherein the at least one perforator is configured to penetrate an epithelium layer of the oral cavity and to deliver the first drug into a submucosa layer.

3. The dissolving SSP patch of claim 1, wherein the at least one perforator is made of a dissolvable biocompatible material configured to pierce the epithelium layer and provide bio-adhesion to the oral cavity.

4. The dissolving SSP patch of claim 1, wherein the at least one perforator is fabricated with a sodium carboxy methyl cellulose (Na-CMC).

5. The dissolving SSP patch of claim 1, wherein the dissolving SSP patch includes about 20 to 100 perforators substantially in an area of 1 cm².

6. The dissolving SSP patch of claim 1, wherein the at least one perforator is configured to with an elongated structure configured in length to penetrate the outside layer of the oral cavity, to deliver the first drug to blood vessels in a submucosa layer, and to prevent damaging the blood vessels in the submucosa layer.

7. The dissolving SSP patch of claim 1, wherein a length of the at least one perforator is substantially in a range of 700 to 1800 μm.

8. The dissolving SSP patch of claim 1, wherein a length of the at least one perforator is shorter than 2000 μm.

9. The dissolving SSP patch of claim 1, wherein the at least one perforator is configured to adhere to the oral cavity until the at least one perforator is substantially dissolved and the first drug is substantially delivered.

10. The dissolving SSP patch of claim 1, wherein the first drug includes an antimigraine drug.

11. The dissolving SSP patch of claim 1, wherein the first drug includes at least one of triptans and dihydroergotamine (DHE).

12. The dissolving SSP patch of claim 11, wherein the first drug includes at least one of analgesics and anesthetics.

13. The dissolving SSP patch of claim 11, wherein the first drug includes non-steroidal anti-inflammatory drugs (NSAIDs).

14. The dissolving SSP patch of claim 1, wherein the first drug include at least one of fentanyl, sufentanil, oxycodone, hydromorphone, morphine, glucagon, and epinephrine.

15. The dissolving SSP patch of claim 1, further comprising:
a basal layer formed on one end of the at least one perforator and configured to provide instant mucosal adhesion to the oral cavity.

16. The dissolving SSP patch of claim 15, wherein the basal layer is configured to include a reservoir containing a second drug.

17. The dissolving SSP patch of claim 16, wherein the second drug is the same as the first drug.

18. The dissolving SSP patch of claim 16, wherein the second drug is different from the first drug.

19. The dissolving SSP patch of claim 16, wherein the second drug is at least one of a antivirus drug and a anti-bacterial protection drug for suppressing infection.

20. The dissolving SSP patch of claim 15, further comprising:
a backing layer formed on the basal layer to cover one side of the basal layer and configured to protect the at least perforator from local saliva and tongue movement.

21. The dissolving SSP patch of claim 20, wherein the backing layer contains flavor and color components to mask medicine taste.

22. The dissolving SSP patch of claim 20, wherein the backing layer is fabricated with a dissolvable and edible material.

23. The dissolving SSP patch of claim 20, wherein the backing layer is fabricated with a material different from materials of the at least one perforator and the basal layer.

24. The dissolving SSP patch of claim 23, wherein the backing layer dissolves at a rate slower than the rate of the at least one perforator and the basal layer.
